(19) United States
(12) Patent Application Publication
(10) Pub. No.: US 2022/0211342 A1
(43) Pub. Date: Jul. 7, 2022

(54) Method Of Performing Automated Measurements Over Multiple Cardiac Cycles

(71) Applicant: GE Precision Healthcare LLC, Wauwatosa, WI (US)

(72) Inventors: Andrew Gilbert, Oslo (NO); Gunnar Hansen, Vestfold (NO); Svein Arne Aase, Trondheim (NO); Andreas Heimdal, Oslo (NO)

(21) Appl. No.: 17/141,444

(22) Filed: Jan. 5, 2021

(51) Int. Cl. A61B 8/08; A61B 8/00; A61B 8/14

(52) U.S. Cl. CPC A61B 8/0883; A61B 8/463; A61B 8/56; A61B 8/14; A61B 8/54; A61B 8/5223

(57) ABSTRACT

An automated measurement system for an ultrasound and/or echocardiographic imaging system enhances reproducibility of measurement results and accommodates form movement in the measurement of the images by combining measurements across multiple cardiac cycles/multiple echocardiogram images. The automated system provides these benefits by initially selecting the cardiac images/cycles for which valid measurements can be obtained within constraints defined by the automated system. With these selected cycles, the automated system then combines the measurements from the selected cycles into a global measurement for the desired parameter or parameters for the combined cycles. This measurement(s) can then be presented to the operator in conjunction with the image or cycle representation for the cycle best approximating the global measurement results, optionally along with the results for the deviation of the combined measurements in the form of display icons representing the deviation from the illustrated cycle image.

### FIELD OF THE DISCLOSURE

The present disclosure relates generally to medical diagnostic devices, and more particularly to ultrasound and/or echocardiography devices.

### BACKGROUND OF THE DISCLOSURE

An echocardiogram, also sometimes referred to as a diagnostic cardiac ultrasound, is a well-accepted medical test that uses high frequency sound waves (ultrasound) to generate an image of a patient's heart. The echocardiogram uses the sound waves to create images of the heart's chambers, valves, walls, and blood vessels (aorta, arteries, veins) attached to the heart. During an echocardiogram, a probe, referred to as a transducer, is passed over the patient's chest and is used to produce the sound waves that bounce off the structures of the heart and “echo” back to the probe. The detected “echoes” are converted into digital images that may be viewed on a computer display.

To detect these conditions and form the resulting images for display, the most common modes of diagnostic ultrasound imaging include B- and M-modes (used to image internal, physical structure), spectral Doppler, and color flow (the latter two primarily used to image flow characteristics, such as in blood vessels), as disclosed in U.S. Pat. No. 8,469,887, entitled Method And Apparatus For Flow Parameter Imaging, the entirely of which is expressly incorporated herein by reference for all purposes. In the present application, all references to echocardiography and/or echocardiography image refer to processes and/or images obtained using any of these imaging types or modes, e.g., B-mode/M-mode/Spectral Doppler/Color Doppler, etc.

The color flow mode is typically used to detect the velocity of blood flow toward/away from the transducer, and it essentially utilizes the same technique as is used in the spectral Doppler mode. Whereas the spectral Doppler mode displays velocity versus time for a single selected sample volume, color flow mode displays hundreds of adjacent sample volumes simultaneously, all laid over a B-mode image and color-coded to represent each sample volume's velocity.

Measurement of blood flow in the heart and vessels using the Doppler effect is well known. The phase shift of backscattered ultrasound waves may be used to measure the velocity of the backscatterers from tissue or blood. The Doppler shift may be displayed using different colors to represent speed and direction of flow. Alternatively, in power Doppler imaging, the power contained in the returned Doppler signal is displayed.

A B-mode ultrasound image is composed of multiple image scan lines. The brightness of a pixel is based on the intensity of the echo return from the biological tissue being scanned. The outputs of the receive beamformer channels are coherently summed to form a respective pixel intensity value for each sample volume in the object region or volume of interest. These pixel intensity values are log-compressed, scan-converted and then displayed as a B-mode image of the anatomy being scanned.

In addition, ultrasonic scanners for detecting blood flow based on the Doppler effect are well known. Such systems operate by actuating an ultrasonic transducer array to transmit ultrasonic waves into the object and receiving ultrasonic echoes backscattered from the object. The sequence of transmitting waves and receiving echo signals is repeated several times for the same scan line and focal positions. The set of echo signals resulting from identical acquisitions is referred to as an ensemble. Since the ensemble is comprised of beams with identical beamforming the only difference among the beams is the information about the position of the scatterers. Position changes of the scatterers translate into phase shifts in the received signals. The phase shifts further translate into the velocity of the blood flow. The blood velocity is calculated by measuring the phase shift from firing to firing at a specific range gate.

Color flow images are produced by superimposing a color image of the velocity of moving material, such as blood, over a black and white anatomical B-mode image. Typically, color flow mode displays hundreds of adjacent sample volumes simultaneously laid over a B-mode image, each sample volume being color-coded to represent velocity of the moving material inside that sample volume at the time of interrogation.

In other ultrasound scanners, the pulsed or continuous wave Doppler waveform is also computed and displayed in real-time as a gray-scale spectrogram of velocity versus time with the gray-scale intensity (or color) modulated by the spectral power. The data for each spectral line comprises a multiplicity of frequency data bins for different frequency intervals, the spectral power data in each bin for a respective spectral line being displayed in a respective pixel of a respective column of pixels on the display monitor. Each spectral line represents an instantaneous measurement of blood flow.

Utilizing any of these imaging modes, echocardiograms are used to identify a variety of different heart conditions of patients as well as provide medical personnel information about the structure and functioning of the heart. For example, using an echocardiogram, a medical professional may be able to identify and/or obtain measurements/measurement results relating to one or more of a) the size and shape of the heart; b) the size, thickness, and movement of the heart's walls; c) movement of the heart; d) the heart's pumping strength; e) whether or not the heart valves are working properly; f) whether or not blood is leaking backwards through the heart valves (regurgitation); g) whether the heart valves are too narrow (stenosis); h) whether there is a tumor or infectious grown around the heart valves; i) problems with the outer lining of the heart (the pericardium); j) problems with the large blood vessels that enter and leave the heart; k) blood clots in the chambers of the heart; and l) abnormal holes between the chambers of the heart.

To identify one or more of these issues in the images received by the echocardiogram transducers, previously an operator would view the image and attempt to locate any issues illustrated in the presented images. As an image is obtained for each cardiac cycle (heartbeat), the operator would view each of the images from each cycle to make this determination. In most cases, the operator reviews the images from the individual cycles and selects the image that best illustrates the structure of the heart for making the determination based on the experience of the operator.

With the opinion and experience of the operator being a significant determining factor in the selection of the cycle used for determination of the condition of the patient, there results a significant element of variation in the measurement results due to the selection of the cycle for obtaining the measurements in this manner. Thus, there is a significant issue with regard to the reproducibility of measurement results for echocardiograms using manual cycle selection processes.

To attempt to address the issue of the lack of reproducibility of the echocardiogram measurement results, automated measurement systems have been developed. The automated systems employ automated measurements to the images associated with each cycle in order to normalize or score the images with regard to the degree of normality or abnormality determined to be present within the individual images. For example, an automated system can apply a simple normal or abnormal score to images from cardiac cycles in order to classify the images as being normal or abnormal based upon preset image parameters stored and utilized by the automated system, such as the system employed in US Patent Application Publication No. US2020/0185084, which is expressly incorporated herein by reference in its entirety for all purposes. The operator can then review the images scored as abnormal in order to more quickly assess issues in those images without also having to assess images scored as normal by the automated system.

However, even with automated measurement systems, the reproducibility of results from echocardiogram images is problematic. In many occasions, there is often significant variation in the images due to movement of the patient during the process of obtaining the images, such as a result of the breathing of the patient, movement of the probe, or different reflection from the imaged tissue, among others. This movement necessarily causes an automated measurement system to classify or score the image for that cycle as abnormal due to the movement of the feature of interest out of the plane of the image as a result of the motion during the cardiac cycle. Thus, an operator would still need to review this image due the abnormal score assessed by the automated measurement system, even though only the movement from patient respiration caused the image to be abnormal rather than any actual abnormality in the heart being imaged.

Therefore, it is desirable to develop a measurement system for assessing and classifying echocardiogram images in a manner that provides enhance reproducibility of the results of the measurements, along with the ability to accommodate for movement of the images across multiple cardiac cycles.

### SUMMARY OF THE DISCLOSURE

According to one aspect of an exemplary embodiment of the invention, an automated measurement system for an ultrasound and/or echocardiographic imaging system is provided that enhances reproducibility of measurement results and accommodates form movement in the measurement of the images by combining measurements across multiple cardiac cycles/multiple echocardiogram images. The echocardiograph image data can include but is not limited to Doppler image data and the echocardiographic images includes but are not limited to images obtained by the ultrasound system operating in one or more of a B-mode (2D/3D)/M-mode/Spectral Doppler/Color mode Doppler. The automated system provides these benefits by initially selecting the cardiac images/cycles for which valid measurements can be obtained within constraints defined by the automated system. With these selected cycles, the automated system then combines the measurements from the selected cycles into a global measurement for the desire parameter or parameters for the combined cycles. This measurement(s) can then be presented to the operator in conjunction with the image or cycle representation for the cycle best approximating the global measurement results, optionally along with the results for the deviation of the combined measurements in the form of display icons representing the deviation from the illustrated cycle image.

According to another aspect of an exemplary embodiment of the invention, the display icons can be varied with respect to one another in order to reflect the confidence in the measurements represented by the various icons.

According to still a further aspect of an exemplary embodiment of the invention, a method of performing an automated echocardiography measurement across multiple cardiac cycles includes the steps of providing an ultrasound imaging system including a control panel including a processing device configured to process ultrasound image data, and an electronic storage device containing algorithms for access and utilization by the processing device, a display operably connected to the control panel, and a transducer operably connected to the control panel to obtain and transmit ultrasound and echocardiograph image data to the control panel, obtaining a number of echocardiographic images over multiple cardiac cycles, selecting a subset of the electrocardiographic images, calculating a global measurement from one or more measured parameters of the subset of the electrocardiographic images and displaying the global measurement.

According to still a further aspect of an exemplary embodiment of the invention, an ultrasound imaging system for performing automated measurements from echocardiographic images obtained over a number of cardiac cycles includes a control panel including a processing device configured to process ultrasound image data and an electronic storage device operably connected to the processing device, a display operably connected to the control panel and a transducer operably connected to the control panel to obtain and transmit ultrasound image data to the control panel, wherein the processing device is configured to employ one or more automated measurement algorithms stored in the electronic storage device to select a subset of echocardiographic images from a number of echocardiographic images obtained by the transducer over multiple cardiac cycles based on one or more of an image quality value, a confidence metric, or combination thereof, and to calculate and display a global measurement of one or more parameters from the subset of echocardiographic images.

These and other exemplary aspects, features and advantages of the invention will be made apparent from the following detailed description taken together with the drawing figures.

### BRIEF DESCRIPTION OF THE DRAWINGS

The drawings illustrate the best mode currently contemplated of practicing the present invention.

In the drawings:

FIG. 1 is a schematic view of an echocardiogram imaging system according to an exemplary embodiment the present disclosure.

FIGS. 2A and 2B are illustrations of echocardiographic images taken over multiple cardiac cycles in a spectral Doppler imaging mode.

FIGS. 3A and 3B are illustrations of the display of the global measurement for the multiple cardiac cycles obtained in a spectral Doppler imaging mode including indicators of measurements for individual cycles.

FIGS. 4A and 4B are illustrations of the display of the global measurement for the multiple cardiac cycles obtained in a spectral Doppler imaging mode including alternative embodiments of the indicators of measurements for individual cycles.

### DETAILED DESCRIPTION OF THE DRAWINGS

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

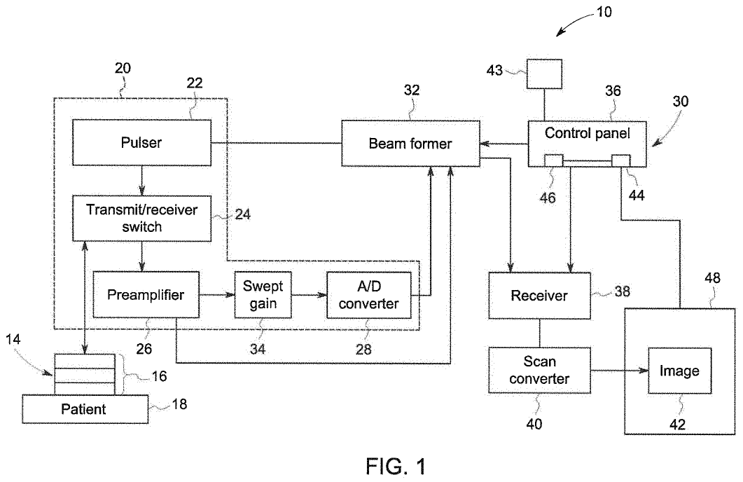

FIG. 1 depicts a high-level view of components of an ultrasound and/or echocardiographic imaging system 10 capable of producing 2D or 3D images, including but not limited to images obtained in a spectral Doppler imaging mode of the selected area of a patient that may be suitable in the implementation of the present approach. In particular, the present approach may be implemented as one or more executable routines and/or algorithms stored on memory or data storage components/databases of the system 10 (such as present in the control panel 36) and/or by one or more application specific integrated circuits (ASICs) of the system 10. The illustrated ultrasound system 10 includes a transducer array 14 having transducer elements 16 suitable for contact with a subject or patient 18 during a cardiac imaging procedure. It should be noted that the transducer array 14 is configured as a two-way transducer and capable of transmitting ultrasound waves into and receiving such energy from the subject or patient 18. In transmission mode, the transducer array elements 16 convert the electrical energy into ultrasound waves and transmit it into the patient 18. In reception mode, the transducer array elements 16 convert the ultrasound energy received from the patient 18 (backscattered waves) into electrical signals.

Each transducer element 16 is associated with respective transducer circuitry 20. That is, in the illustrated embodiment, each transducer element 16 in the array 14 has a pulser 22, a transmit/receive switch 24, a preamplifier 26, a swept gain 34, and an analog to digital (A/D) converter 28. In other implementations, this arrangement may be simplified or otherwise changed. For example, components shown in the circuitry 20 may be provided upstream or downstream of the depicted arrangement; however, the basic functionality depicted will typically still be provided for each transducer element 16.

Further, a variety of other imaging components 30 are provided to enable image formation with the ultrasound system 10. Specifically, the depicted example of an ultrasound system 10 also includes a beam former 32, a control panel 36, a receiver 38, and a scan converter 40 that cooperate with the transducer circuitry 20 to produce an image or series/number of echocardiographic images 42 (e.g., an echocardiogram) that may be stored and/or displayed to an operator. A processing component 44 (e.g., a microprocessor) and an electronic storage device or database 46 of the system 10, such as present in control panel 36, may be used to execute stored routines for processing the acquired ultrasound cardiac images to generate various measurements, other information and/or motion frames, as discussed herein, which may be displayed on a monitor 48 of the ultrasound system 10.

In the method of operation, the transducer array or probe 14 including the transducer elements 16 is placed against the patient 18 and operated to acquire the ultrasound cardiac images 42. There are typically several cardiac cycles (i.e., 1-30 cycles) that occur during each echocardiography acquisition. Based on movements of the probe 14, movement of the patient 18 i.e., respiration, or different reflections from there is often some variability in the measurement obtained by the probe 14 across the cardiac cycles that occurred during the acquisition. In order to address this variability in the measurements, the system 10 utilizes the processing component or device 44 to combine the computed measurements across multiple cardiac cycles using automated measurement algorithms contained within the electronic storage medium/database 46 and utilized by the processing unit 44 and subsequently visualizing these results to the user.

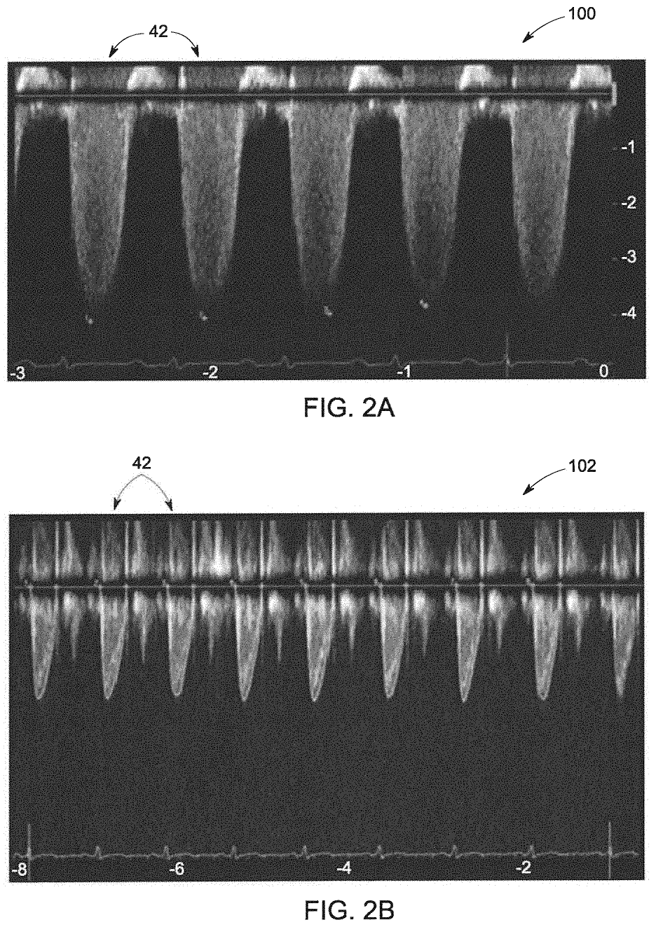

In the method for performing the automated measurement determination over the cardiac cycles that occurred during the ultrasound image acquisition, the first step is selection by the processing device 44 of the recorded cardiac cycles to be utilized in performing the automated measurement. During the acquisition process some images in certain cardiac cycles may not be suitable for use in calculating the desired measurements for the acquisition for a number of reasons, including as a result of the user switching between two views in a single acquisition, or because respiration of the patient caused the feature of interest to leave the plane of the image in certain cycles, among others. Examples of the images 100, 102 obtained in a spectral Doppler imaging mode and provided for the measurements obtained for each of a number of cardiac cycles/images 42 and the variation in these images 42 is shown in FIGS. 2A and 2B. With the variation in the cardiac cycles created by these and/or other causes, in many cases only a subset of the echocardiographic images/cardiac cycles 42 can be used/provide accurate measurement information. As such, it is important for the processing device 44 to be able to select only those echocardiographic images/cardiac cycles 42 for use in the calculation of the desired measurement that provide accurate data or one or more parameters for the measurement determination.

In one exemplary embodiment, the cycle selection step is performed by a view recognition algorithm employed by the processing device 44. In the employment of the view recognition algorithm, only those images 42/cardiac cycles which produce a stable classification result for the view of the image with a high degree of confidence are included in the image set utilized for the measurement calculation. For example, in this review or selection process by the processing device 44, for cycles/images 42 where respiration of the patient 18 caused the feature of interest to temporarily move out of the plane of the image 42, the selection step enables the processing device 44 to automatically discard those cycles/images 42 from those utilized in the determination of the measurement result(s). The view recognition algorithm detects the view of the heart obtained during the individual cardiac cycle, such as a 2-chamber view or a 4-chamber view, in order to determine whether the view matches the desired view (e.g., either the 2 chamber view or the 4 chamber view) for obtaining the desired measurement information. In one particular example, the desired view utilized by the view recognition algorithm can be a view that is directly centered on the apex of the left ventricle, in order to prevent images suffering from foreshortening from being utilized in the measurement determination, with the view recognition algorithm also providing a confidence metric for each reviewed cardiac cycle/image 42 that the particular cycle/image 42 corresponds to the desired view. Upon a determination by the processing device 44 via the view recognition algorithm that the view for a particular cardiac cycle corresponds to the desired view for the measurement calculation, the processing device 44 can include the particular image for use in the measurement calculation.

In another exemplary embodiment for the cycle selection step, the selection of the cardiac cycles to be utilized in the measurement calculation is performed by a network that detects image quality. In this embodiment, the cycle selection could be made by any algorithm that can determine image quality, e.g, an image quality confidence metric. One particular example would then be a neural network that is trained to classify images based on the perceived quality as determined by a clinical expert to determine the image quality metric. For making the image quality determination, a number of different parameters can be utilized by the network/image quality algorithm, such as the brightness of the image, the acoustical impedance of the image, visibility of important structures, or others, either alone or in combination with one another. In this embodiment, the network or image quality algorithm reviews the images 42 for each cardiac cycle to determine the image quality for each cycle where only the cycles having images of a minimum quality are included for the measurement determination.

In still another exemplary embodiment, the selection step can be performed using a confidence metric for each cardiac cycle image 42 that provides an indication of how expected and/or “normal” the measurement result(s) is for the specific cardiac cycle, e.g., a measurement variance confidence metric. The confidence metric can have various forms and can come from different sources. For example, the confidence metric can be extracted from or determined in relation to the output of the automated measurement algorithm itself. For example, a confidence metric can be based on a comparison of the measurement for a particular cardiac cycle/image as determined by the automated measurement algorithm, with the other measurement values determined for other cardiac cycles or to correlated measurements on the same patient, for example. The confidence metric can be based on the magnitude of any difference between the calculated measurement and the compared measurement value, and any images/cardiac cycles falling outside of an acceptable range around the predetermined value can be discarded from the final measurement determination. Alternatively, as is the case with many deep learning algorithms, another algorithm in the processing step for the measurement, such as the view recognition algorithm, for example, can be utilized for the determination of the confidence metric/value for each cardiac cycle/image. In addition, specifically when the algorithm utilized is a neural network, other examples of the algorithms that can be used in the determination of the confidence metric include but are not limited to: a) a network that outputs the prediction as a distribution of possible measurements, b) an network which outputs the prediction and separately a measure of confidence, or c) a network that is used to process the same image multiple times, but with slightly varying parameters during each processing sequence, where the confidence metric is arrived at using the variance of the outputs/measurement result(s) across those processing runs.

In still another exemplary embodiment, the selection step can be performed by the processing device 44 utilizing some combination of one or more of the prior described embodiments for the selection step or process, such as by utilizing the view recognition confidence metric in association with the image quality metric, and/or the measurement variance confidence metric.

After the cycle selection step is performed by the processing device 44, the cardiac cycle/images 42 that were selected are employed in a second step of determining or calculating a single global measurement for the desired parameter of each of the selected cardiac cycles. This global measurement can be determined in a variety of acceptable manners, but in one exemplary embodiment is determined by averaging the selected cardiac cycles/measured parameter(s) of each cycle with one another. This calculation can be performed either as a simple mean/median of the cycles/cycle parameter(s) or a weighted average where the weighting comes from any of the methods used for cycle selection step, e.g., where those cardiac cycles/images having higher image confidence and/or quality values, and/or higher confidence metrics are given greater corresponding weights in the averaging.

In addition, a measure of global measurement validity or confidence in the averaged global measurement itself can be provided along with the global measurement for all of the selected cycles. In one exemplary embodiment, the global measurement validity or confidence value can be determined utilizing one or both of the confidence metrics extracted from each individual cycle, or the variance of the individual measurements from one another across the selected cycles which can additionally each also weighted by the confidence metric for the individual cycle. Further, while the global measurement validity value can be displayed along with the global measurement in all cases, in certain cases where the global measurement results from only a single cycle that can be shown to the user, e.g. 2D measurements, either the median result or the confidence metric can be used to determine which cycle/image 42 will be presented on the display 48.

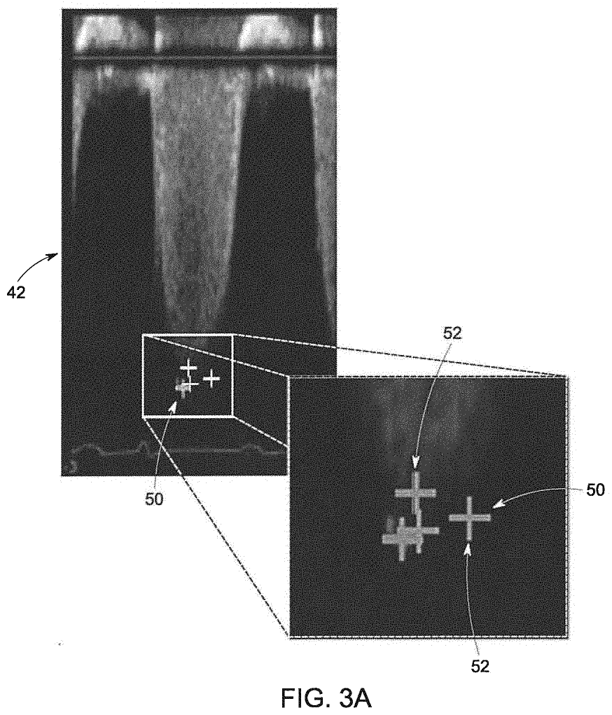

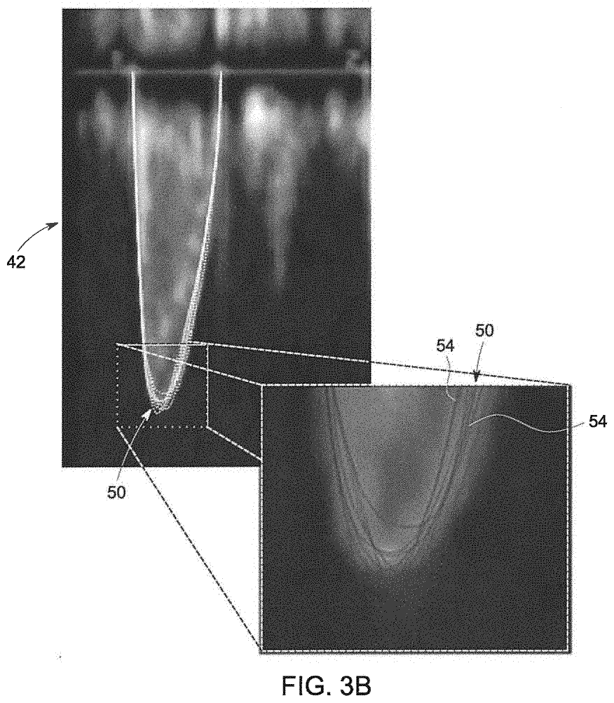

Looking now at FIGS. 3A and 3B, after calculating the global measurement and optionally the global measurement validity, from the cardiac cycles/images 42 selected in the first step of the method, the results of this analysis by the processing device 44 can be provided to the user on the display 48. In doing so, the processing device 44 determines the cycle image 42 of those selected in the first step having the highest confidence metric and/or the highest image quality selected by the processing device 44 in the selection step and utilized in the determination of the global measurement. This cycle image 42 is presented on the display 48 along with calipers or indicators 50 that illustrate the measurement results across the various cycles selected for the determination of the global measurement. In this manner, in addition to showing the global measurement result(s), including but not limited to, e.g., the mean/standard deviation across all selected cycles/images 42, and the cycle/image 42 best fitting this global measurement, all measurements for each selected cycle/image 42 can be represented and grouped together on the single cycle/image 42 presented in the display 48 using the indicators 50 to easily assess differences between the cycles/images 42.

In FIG. 3A the indicators 50 are shown as icons 52. The icons 52 can have any desired shape and are positioned on the image 42 at locations corresponding to the difference in the values for the individual cycle represented by the icon 52 from that of the presented image 42 on the display 48. Alternatively, as shown in FIG. 3B, the indicators 50 can take the form of image lines 54 that graphically illustrate the position of the cycle/image 42 that the line 54 represents relative to the displayed image 42 for the global measurement. With the location of the icons 52 or lines 54, the user can visually determine the differences between the measurement values for each of the individual cycles/images 42 utilized in the determination of the global measurement, while simultaneously viewing the cycle/image 42 best representing the global measurement, e.g., the image 42 having the highest image confidence value, image quality value, confidence metric, or combination thereof. Additionally, the indicators 50 can be links that can be selected by the user, such as by using a touch screen or mouse (not shown) connected to the control panel 36 system 10 and forming a user input device 43 (FIG. 1), to present that selected image 42 represented by the indicator 50 on the display 48.

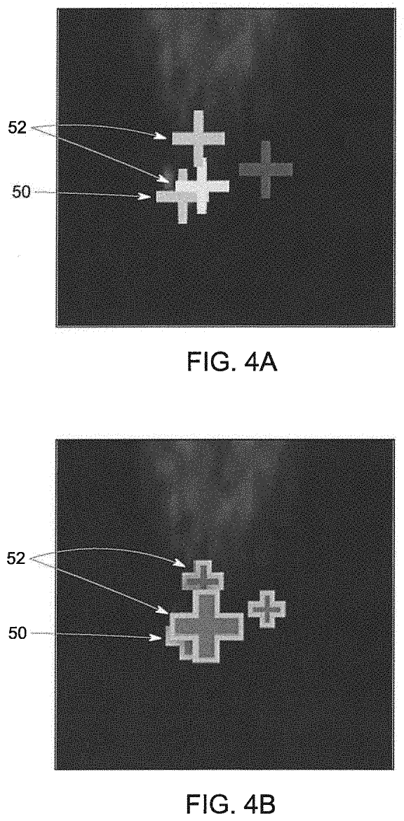

In addition, the indicators 50, e.g., the icons 52 or the lines 54, can be displayed with the indicators 50 having differences relative to one another with regard to their appearance. The differences between the appearance of the individual indicators 50 can represent the confidence metric and/or quality of the image the indicator 50 represents. The differences for the indicators 50 can be selected as desired and can include different colors and/or opacities (FIG. 4A), or different sizes (FIG. 4B) for the indicators 50, among other suitable differentiating attributes. With this representation of the indicators 50 in conjunction with the displayed cycle/image 42 representing the global measurement result(s) and the global measurement result(s), increased confidence in the global measurement is provided based on the ability of the user to readily view the measurements of each individual cycle/image 42 employed in the determination of the global measurement result(s).

According to another exemplary embodiment, as opposed to finding the cycle/image 42 that best fits the global measurement result(s), i.e., that has the highest image confidence value, image quality value, confidence metric, or combination thereof, the processing device 44 can create a simulated cycle/image 42 that fits the global measurement. This simulated cycle/image 42 can be presented on the display 48 along with the indicators 50 representing the measurement values for each of the selected cycles/images 42 used to form the global measurement and the simulated cycle/image 42.

With the ability of the processing device 44 to review multiple images 42 obtained over multiple cardiac cycles, the method employed by the processing device 44 is applicable to any measurement that can be and/or is desired to be calculated or determined across multiple cycles to provide the ability to minimize the variability in the measurement results while concurrently increasing the confidence in and reproducibility of the measurements in a readily presentable and transparent manner.

As such, the method of the present disclosure provides the following benefits with regard to the determination of any measurement, e.g., measurements of the thicknesses of the walls or portions of walls of the heart, the velocity or volume of blood flow through the chambers of the heart and/or any vessels around the heart or other diagnostic measurements used in a standard echocardiography evaluation that is calculated from ultrasound or echocardiographic imaging across multiple cardiac cycles:
1. A method for selecting which cycles to use for automated measurements using any combination of:
   a) view recognition;
   b) automatic quality assessment; and/or
   c) measurement confidence metrics.
2. A method of compounding the measurement values using either:
   a) traditional statistical methods (mean/median); or
   b) a weighted average based on the results of any of the methods from (1).
3. A global measurement variability metric extracted from the weighted variability of the weighted compounding in (2).
4. A method for determining the optimal measurement cycle to show to the user using any of the methods from (1).
5. A method for visualizing measurement variability results to the user.

Before the present compositions, apparatuses and methods are described, it is understood that this invention is not limited to the particular embodiments and methodology, as these may vary. It is also to be understood that the terminology used herein is for the purpose of describing particular exemplary embodiments only, and is not intended to limit the scope of the present invention which will be limited only by the appended claims.

What is claimed is:

1. A method of performing an automated echocardiography measurement across multiple cardiac cycles, the method comprising:
providing an ultrasound imaging system including a control panel including a processing device configured to process ultrasound image data, and an electronic storage device containing algorithms for access and utilization by the processing device, a display operably connected to the control panel, and a transducer operably connected to the control panel to obtain and transmit ultrasound and echocardiograph image data to the control panel;
obtaining a number of echocardiographic images over multiple cardiac cycles;
selecting a subset of the electrocardiographic images;
calculating a global measurement from one or more measured parameters of the subset of the electrocardiographic images; and
displaying the global measurement.

2. A method according to claim 1, wherein the step of selecting the subset of echocardiographic images comprises applying a view recognition algorithm to the number of echocardiographic images to determine the subset of the echocardiographic images which each have a stable classification result for a view of the echocardiographic image with a high degree of confidence.

3. A method according to claim 1, wherein the step of selecting the subset of echocardiographic images comprises applying an image quality algorithm that determines the subset of the echocardiographic images having a minimum image quality.

4. A method according to claim 1, wherein the step of selecting the subset of echocardiographic images comprises applying a confidence metric to each of the number of echocardiographic images.

5. A method according to claim 4, wherein the step of applying the confidence metric comprises comparing a measurement for each of the number of echocardiographic images with a predetermined value for that measurement.

6. A method according to claim 1, wherein the step of selecting the subset of echocardiographic images comprises applying a combination of a view recognition algorithm, an image quality algorithm, and a confidence metric to each of the number of echocardiographic images.

7. A method according to claim 1, wherein the step of calculating the global measurement comprises averaging the one or more measured parameters of each of the subset of echocardiographic images with one another.

8. A method according to claim 7, wherein the step of averaging the one or more measured parameters comprises determining a simple mean/median of the one or more parameters.

9. A method according to claim 7, wherein the step of averaging the one or more measured parameters comprises determining a weighted average of the one or more parameters.

10. A method according to claim 9, wherein the step of determining the weighted average of the one or more parameters further comprises the steps of:
applying a weight to the one or more parameters for each of the subset of echocardiographic images, wherein the weight for the one or more parameters of each of the subset of echocardiographic images is obtained from results of the step of selecting the subset of echocardiographic images; and
averaging the weighted one or more parameters for each of the subset of echocardiographic images.

11. A method according to claim 10, wherein the weight for the one or more parameters of each of the subset of echocardiographic images corresponds to a value for each of the subset of echocardiographic images selected from the group of: an image confidence value, and image quality values and a confidence metric.

12. A method according to claim 1, further comprising the steps of:
determining a global measurement confidence value after selecting a subset of the electrocardiographic images; and
displaying the global measurement confidence value with the global measurement.

13. A method according to claim 12, wherein the step of determining the global measurement confidence value comprises comparing the confidence metrics for each of the subset of echocardiographic images to a reference value.

14. A method according to claim 12, wherein the step of determining the global measurement confidence value comprises determining the variance of the one or more parameters across the subset of echocardiographic images.

15. A method according to claim 1, wherein the step of displaying the global measurement comprises:
displaying a single echocardiographic image; and
displaying a number of indicators in association with the single echocardiographic image.

16. A method according to claim 15, wherein the single echocardiographic image is an echocardiographic image selected from the subset of echocardiographic images having the highest image confidence value, image quality value, confidence metric, or combination thereof.

17. A method according to claim 15, wherein the single echocardiographic image is a simulated echocardiographic image generated by the processing device corresponding directly to the global measurement of the one or more parameters.

18. A method according to claim 15, wherein the number of indicators each represent measurement values for the one or more parameters of each of the subset of echocardiographic images relative to the global measurement.

19. A method according to claim 15, wherein the number of indicators each have appearances corresponding at least one of: the confidence metric of each of the echocardiographic images of the subset of echocardiographic images, or the variation of the measurement of the one or more parameters of each of the subset of echocardiographic images relative to the global measurement.

20. An ultrasound imaging system for performing automated measurements from echocardiographic images obtained over a number of cardiac cycles, the echocardiographic imaging system comprising:
a control panel including a processing device configured to process ultrasound image data and an electronic storage device operably connected to the processing device;
a display operably connected to the control panel; and
a transducer operably connected to the control panel to obtain and transmit ultrasound image data to the control panel,
wherein the processing device is configured to employ one or more automated measurement algorithms stored in the electronic storage device to select a subset of echocardiographic images from a number of echocardiographic images obtained by the transducer over multiple cardiac cycles based on one or more of an image confidence value, an image quality value, a confidence metric, or combination thereof, and to calculate and display a global measurement of one or more parameters from the subset of echocardiographic images.
